(19) United States
(12) Patent Application Publication
(10) Pub. No.: US 2025/0223708 A1
(43) Pub. Date: Jul. 10, 2025

(54) METHOD FOR ELECTROCHEMICAL REDUCTION OF LIQUID OR SUPERCRITICAL CO2

(71) Applicant: Mickaël ARNOLD, Sisteron (FR)

(72) Inventor: Mickaël ARNOLD, Sisteron (FR)

(21) Appl. No.: 18/724,197

(22) PCT Filed: Dec. 29, 2022
(86) PCT No.: PCT/EP2022/088042

§ 371 Date: Jun. 26, 2024

(30) Foreign Application Priority Data
Dec. 31, 2021 (FR) FR2114745

(51) Int. Cl. C25B 1/23; C07C 1/04; C25B 3/26; C25B 9/17; C25B 9/19; C25B 9/70; C25B 15/08

(52) U.S. Cl. CPC C25B 1/23; C07C 1/0495; C25B 3/26; C25B 9/17; C25B 9/19; C25B 9/70; C25B 15/081

(57) ABSTRACT

The invention relates to a method for the electrochemical reduction of carbon dioxide in the liquid or supercritical state, comprising at least two electrodes separated from each other by a distance of less than or equal to 7 millimeters, preferably less than or equal to 1 millimeter.

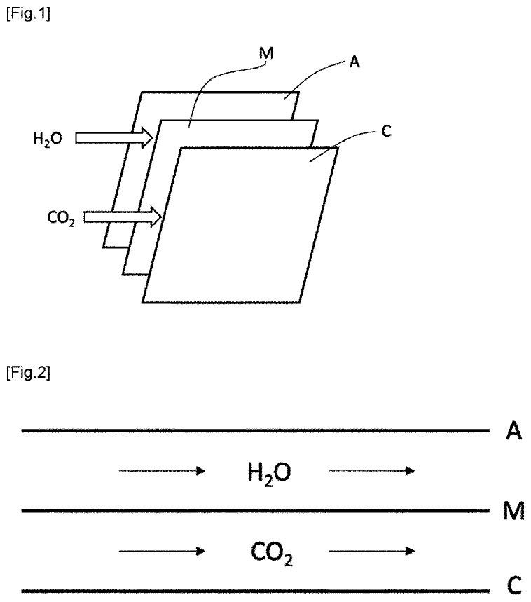

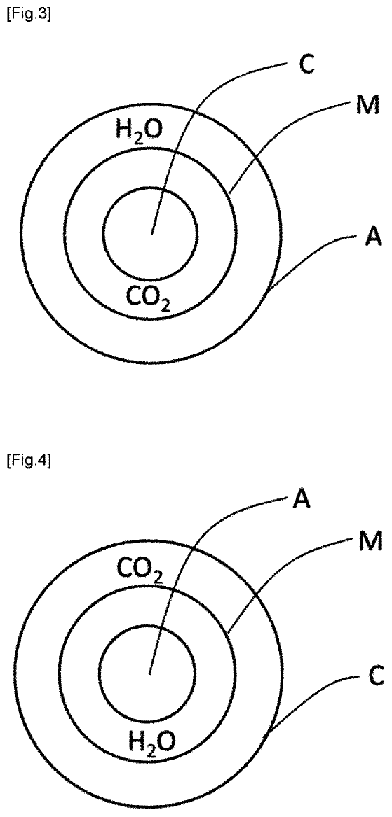

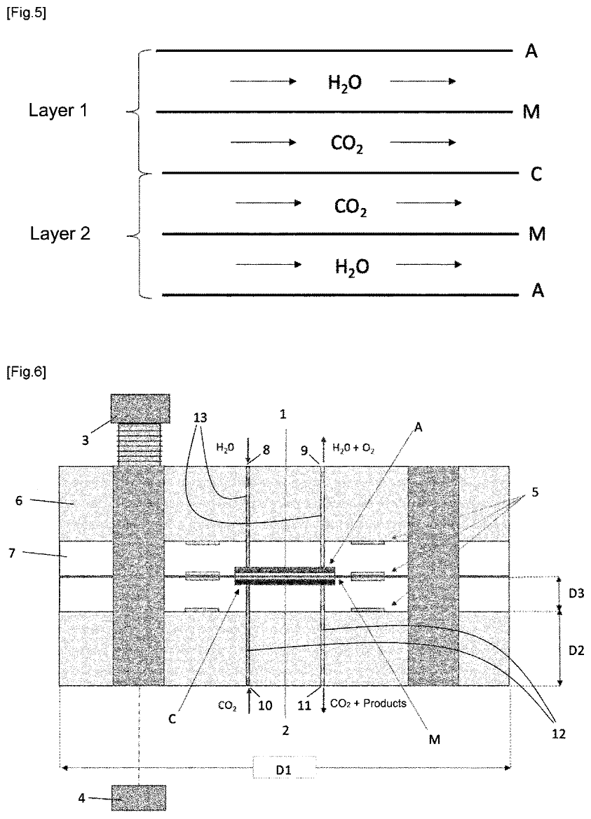

### The invention relates to a method, preferentially industrial, for the electrochemical reduction of carbon dioxide and to an electrochemical reactor.

### INTRODUCTION

Carbon dioxide results from carbon oxidation processes, such as respiration, combustion, and so on, which release energy. Respiration consists, essentially, of breaking down glucose or fats. Additionally, it is an oxidation process (combustion) that humans use for heat and mobility when burning wood or fossil fuels such as coal, oil or natural gas. Fossil fuel combustion in human activities such as transport, petrochemical manufacturing, electricity generation, etc. produces billions of tons of carbon dioxide every year. By way of illustration, in two centuries (i.e. since the industrial revolution), the carbon dioxide content of the atmosphere has risen from 278 ppm to 400 ppm, an increase of 40%. And yet, as is well known, rising atmospheric carbon dioxide levels are responsible for damaging effects on the planet, such as climate disruption and pH changes in the seas and oceans.

One possible way to mitigate carbon dioxide emissions is to convert carbon dioxide into economically useful materials, such as fuels and industrial chemicals. The increasing amount of carbon dioxide in the atmosphere makes the latter particularly attractive for conversion into value-added products. Thus, ideally, it would be interesting to have an economically viable technical means for 1) drawing on this resource (i.e. atmospheric CO₂), and 2) using the CO₂ as a raw material of interest, in particular for producing energy products or at least industrial useful products.

Thus, the reduction of CO₂ into combustible materials (hydrocarbons, alcohol, graphite, etc.) has the advantage of offering non-polluted combustions, for which CO₂ recovery is easy. Thus, the CO₂ is recycled, sometimes as a reaction product, sometimes as a raw material to be reduced.

Carbon dioxide (CO₂) is a stable molecule which is the most oxidized form of carbon. As the end product of combustion, CO₂ has high thermodynamic stability. As a result, many methods for converting CO2 into useful end products are energy-intensive and/or hazardous.

Converting CO₂ into more reduced forms of carbon therefore requires an energy input. Photosynthesis is a biological process capable of carrying out this conversion.

Photosynthesis uses light energy to transform CO₂ and water (H₂O) into carbohydrates [—CH₂O—] such as glucose (C₆H₁₂O₆). These carbohydrates are used as energy reserves and basic materials for the synthesis of organic matter. Photosynthesis thus transforms light energy into chemical energy. This chemical energy is stored in the form of C—C and C—H bonds by CO₂ reduction. Plant organic matter is therefore not only a reserve of matter, but also of energy. Over time, biological and physical-chemical processes have transformed this organic plant matter into fossil fuels: coal, oil and gas.

However, the process of photosynthesis is relatively slow and therefore requires a large surface area (such as forests) to meet the current energy needs of humanity.

Alternatively, the thermodynamic stability of CO₂ can be circumvented by a simple one-electron reduction at an electrode, leading to the in situ generation of reactive intermediates.

An interesting alternative conversion method involves the electrochemical reduction of CO₂. With electricity increasingly coming from renewable sources, organic electrosynthesis appears to be a promising technology for environmentally-friendly chemical methods, with capacity to meet current and future energy needs, at least periodically.

Thus, the electrochemical reduction of CO₂ can be applied to the synthesis of fuels such as formic acid, methanol or methane. Carbon in its reduced forms is therefore a potential source of energy, whether in simple molecules like methanol or methane, or in more complex molecules like glucose or hydrocarbons. This method has the added advantage of enabling periodic storage of fuels, for example, for momentarily increased electricity production. This strategy thus belongs to an eco-responsible approach to managing means for energy production.

Several CO₂ reduction reactors are known. The CO₂ reduction reactor configurations have in common the use of an ion-selective membrane of the cation or anion exchange type, and the use of an aqueous phase wherein the CO₂ is solubilized in the cathode compartment.

Thus, the reduction and the oxidation take place in the cathodic and anodic compartments of the electrochemical reactor, respectively.

Additionally, catalytically active bipolar membrane technology can contribute to electrochemical conversions (cf. Balster et al., Chemical Engineering and Processing 2004, 43, 1115-1127).

A bipolar membrane is a synthetic membrane comprising two ion exchange layers of opposite charge in contact with each other. Thus, a bipolar membrane can be seen as the combination of a cation exchange membrane and an anion exchange membrane. Bipolar membranes are known to be part of the design of electrodialysis cells. Because of this arrangement of charged layers, the bipolar membrane is not effective for transporting cations or anions across the entire width of the membrane, and must be distinguished from the ion-selective membranes used in conventional electrochemical reduction of CO₂.

The use of a bipolar membrane in the electrochemical reduction of CO₂ is described in document CN102912374. CN102912374 relates to an electrolytic reservoir for the electrochemical reduction of CO₂ using a bipolar membrane as a diaphragm and to an application of the electrolytic reservoir for the electrochemical reduction of CO₂. The electrochemical/electrolytic reduction of CO₂ in this document involves ambient temperature and pressure.

WO2014/043651 and US20130105304 also describe a method for the electrochemical reduction of CO₂ at high pressure. In the methods disclosed in these documents, the CO₂ is dissolved in water, which does not allow high CO₂ densities to be achieved in the reactor, thus de facto limiting the yield.

WO2019010095A1 discloses methods for producing alcohols or methane by drawing CO₂ from the air or from another dilute source, and by supplying water, which is converted into hydrogen and oxygen.

The scientific papers by Y. Hori et al. (Electrochimica Acta, Vol. 39, No. 11/12, pages 1833-1839, 1994) and T. Saeki et al. (Journal of Electroanalytical Chemistry 390, (1995), 77-82) also provide insights into CO₂ reduction in the presence of methanol or water.

U.S. Pat. No. 9,469,910B2 discloses a method for producing hydrocarbons from CO₂ and water, using electrolysis and two separate reactors.

In WO2017014635, CO₂ is the main reaction medium (i.e. the solvent) and a small fraction of water is added to form ionic reaction species on the cathode side. It is disclosed that these ionic reactive species ensure electrical conductivity and participate (simultaneously) in the overall carbon dioxide reduction reaction, and that these two aspects (conductivity and participation in the reaction) enable minimal use, or even no use, of an electrolyte/catholyte solution.

Typically, the methods described hereinbefore make it possible to reduce CO₂. However, the techniques are generally too complex to be industrialized. For example, the systematic presence of membranes and/or electrolytes makes the methods too restrictive (in terms of inspections to be carried out, for example), and/or too hazardous to be adapted to the industrial scale.

The subject matter of the present invention is to overcome one or more of the problems encountered in the prior art.

### SUMMARY OF THE INVENTION

The subject matter of the present invention relates to a method for the electrochemical reduction of carbon dioxide, which is in the liquid or supercritical state.

A first subject of the present invention thus relates to a method for the electrochemical reduction of carbon dioxide in the liquid or supercritical state, comprising at least two electrodes separated from each other by a distance of less than or equal to 7 millimeters (mm), preferentially less than or equal to 1 mm.

The reduced product(s) obtained by the method according to the present invention, such as carbon monoxide (CO), can thus be reaction intermediates used in other subsequent reactions. For example, the CO obtained by the method according to the present invention can then be used in a Fischer-Tropsch reaction or in a condensation in order to create a carbon-carbon bond.

Thus, electrochemistry is used as a technique and energy source for CO₂ reduction. The conventional overall mechanism consists in reducing the CO₂ to CO at the cathode (i.e. the electrode that receives electrons from the generator) and oxidation at the anode (i.e. the electrode that donates electrons to the generator). However, it has been discovered that the distance between the two types of electrodes (cathode(s) and anode(s)) does away with the need for electrolyte, catalyst and/or ion exchange membrane.

The CO₂ entering the method is in the supercritical or liquid state. Preferably, the CO₂ entering the method is exclusively in the supercritical state. Thus, the method of the present invention preferentially comprises a first step that consists in converting the CO₂ gas into supercritical or liquid CO₂. This step is carried out by compression using a suitable pump.

The method of the present invention can be carried out in the absence of water or in the presence of water, which is then a proton donor. Other proton donors are also conceivable in the context of the present invention.

In the particular case, wherein CO₂ is the only reagent (i.e. there is no reducing agent), the method of the invention produces CO and O₂.

When the method of the invention uses water, which is then a proton donor, the water breaks down at the anode (positive pole (+) of the generator) to donate H⁺ protons according to the equation: H₂O→1/2 O₂+2H⁺+2^{e-}. The CO produced during the method of the invention can, in turn, be used in a methanol synthesis reaction or in a Fischer-Tropsch reaction to produce hydrocarbons.

A second subject of the present invention relates to a reactor, preferentially an industrial reactor, for carrying out the method for the electrochemical reduction of carbon dioxide according to the present invention. Such a reactor comprises at least two electrodes separated from each other by a distance of less than or equal to 7 mm.

A third subject of the present invention relates to a method for synthesizing methanol and/or at least one hydrocarbon, consisting in carrying out a Fischer-Tropsch reaction from carbon monoxide (CO) and hydrogen (H₂), the CO being obtained according to the method for the electrochemical reduction of CO₂ described herein.

A fourth subject of the present invention also relates to an industrial reactor complement, or to a second reactor positioned in series with the first, for carrying out the method for the electrochemical reduction of carbon dioxide according to the present invention for carrying out the method for synthesizing methanol and/or at least one hydrocarbon, consisting in carrying out a Fischer-Tropsch reaction from carbon monoxide (CO) obtained according to the method for the electrochemical reduction of carbon dioxide (CO₂) described herein.

A fifth subject of the present invention relates to a reaction device comprising at least one reactor for carrying out the method for the electrochemical reduction of carbon dioxide according to the present invention, for example comprising one or more reactor(s) arranged in series. The reaction device may, additionally, comprise an industrial reactor complement.

### DEFINITIONS

In the context of the present invention, the term “carbon dioxide in the liquid or supercritical state” refers to any liquid form comprising carbon dioxide, also referred to equivalently in the context of the present invention as a “liquid formulation comprising carbon dioxide”. Preferably, the carbon dioxide in the liquid or supercritical state comprises predominantly carbon dioxide, i.e. at least 50% by weight of carbon dioxide with respect to the total weight of the liquid comprising the carbon dioxide. The term “supercritical carbon dioxide” refers to a fluid state of carbon dioxide, obtained when it is maintained above its critical temperature and pressure: 304.25 K and 72.9 atm respectively.

The present invention belongs to the context of “milli-fluidics” or “micro-fluidics”, i.e. a system handling fluids in milli-channels or micro-channels, of millimetric or micrometric size (of the order of a thousandth of a millimeter), respectively, and which enables the manufacture of devices handling very small amounts of liquid in milli-volumes or in micro-volumes.

The expression “at least two electrodes separated from each other by a distance x” indicates a distance x between the surfaces of each electrode. In other words, the surface of one electrode is separated from the nearest surface of the neighboring electrode by the indicated distance x.

In the context of the present invention, the term “electrolyte” refers to a conductive solution (thus, in liquid form) in which salts, also referred to as conductive molecules, are dissolved, and which conducts electric current. In the context of the present invention, a distinction is made between a “solvent” and an “electrolyte”. A solvent is described as an electrolyte when conductive molecules are added to it (or dissolved therein). Thus, water as such (without the addition of conductive molecules) does not constitute an electrolyte for the purposes of the present invention. Similarly, methanol or ethanol as such (without the addition of conductive molecules) do not constitute an electrolyte for the purposes of the present invention.

The term “ion exchange membrane” refers to membranes, advantageously impermeable to gases, made of the same materials as the ion exchange resins used in separative techniques. The ion exchange membrane used in the present invention can be of the monofunctional or homopolar type, or alternatively of the bi-functional type, in which ion exchange sites of different natures coexist. Preferably, the ion exchange membrane that can be used in the context of the present invention is a proton exchange membrane, advantageously impermeable to gases. When using a membrane reactor, the proton exchange membrane (PEM) can be separated from the electrodes by meshes or fabrics.

“Fischer-Tropsch reaction” refers to the reaction involving the catalytic reduction (typically heterogeneous) of carbon monoxide (CO) by hydrogen (H₂) to convert them into hydrocarbons. Catalysts such as iron, cobalt, ruthenium or even nickel can, for example, be used to drive this reaction. Fischer-Tropsch synthesis can thus consist in synthesizing (for example at pressures greater than 50 bar, at temperatures greater than 150° C. and with the aid of catalysts) hydrocarbons from carbon (CO) and hydrogen (H₂) according to the following equation: (2n+1)H₂+n CO→CₙH₂ₙ₊₂+nH₂O.

In the context of the present invention, the term “industrial reactor” means a device for carrying out chemical reactions on an industrial scale, i.e. allowing the production of industrial amounts of products resulting from chemical reactions. Such amounts of product(s) obtained may be greater than or equal to 10 kg, greater than or equal to 25 kg, greater than or equal to 50 kg, greater than or equal to 100 kg, greater than or equal to 500 kg, greater than or equal to 1,000 kg, greater than or equal to 5,000 kg, or even greater than or equal to 10,000 kg, per day.

“Reactor positioned in series” means a second industrial reactor positioned at the outlet of a first industrial reactor, so that the products from the first industrial reactor (possibly purified) are introduced directly into the second industrial reactor and then considered to be reagents in a second reaction taking place in the second industrial reactor.

In the context of the present invention, the term “industrial reactor complement” refers to a complementary device of an industrial reactor as defined hereinbefore.

### DETAILED DESCRIPTION

In the context of the present invention:
the expression “comprised between . . . and . . . ” (for example, a range of values) should be understood as including the limits (for example, the limit values of this range of values);
any description connected with one embodiment is applicable and interchangeable with all other embodiments of the invention; and
when a feature or component is included in and/or selected from a list of features or components, it must be understood that this individual feature or component may be selected and combined with other individual features, or may be selected to form a subgroup of two or more explicitly listed features or components; likewise, any feature or component cited in a list of features or components may be omitted from that list.

A first subject of the present invention relates to a method for the electrochemical reduction of carbon dioxide in the liquid or supercritical state, comprising at least two electrodes separated from each other by a distance of less than or equal to 7 millimeters (mm). In other words, the present invention relates to a method for the electrochemical reduction of carbon dioxide in the liquid or supercritical state, carried out within a reaction device comprising at least two electrodes separated from each other by a distance of less than or equal to 7 mm. Preferentially, the CO₂ is in supercritical form (i.e. 31.06° C. and 73.83 bar).

In the context of the present invention, the at least two electrodes are separated by a distance of less than or equal to 7 mm, preferentially less than or equal to 5 mm, less than or equal to 3 mm or less than or equal to 1 mm. For example, the at least two electrodes are separated from each other by a distance of less than or equal to 900 micrometers (μm), less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 100 μm, such as less than or equal to 50 μm (e.g. about 40 μm in the absence of an ion exchange membrane).

The reduction method of the present invention comprises at least two

electrodes, i.e. it can comprise two or more electrodes. For example, it may comprise between 2 and 100 electrodes, for example between 2 and 80 electrodes or between 2 and 50 electrodes.

According to one embodiment, the method for the electrochemical reduction of carbon dioxide in the liquid or supercritical state of the present invention is carried out within a continuous flow reaction device, for example a continuous (flow) reactor. An example of a continuous (flow) reactor is a planar or tubular reactor. When such a type of reaction device or continuous reactor is used, the method of the invention is referred to as a milli-fluidic or micro-fluidic method for the electrochemical reduction of CO₂.

According to one embodiment, the CO₂ in the liquid or supercritical state comprises predominantly CO₂, i.e. at least 50% by weight of CO₂ with respect to the total weight of the liquid comprising the CO₂. In other words, according to this embodiment, the liquid formulation comprising the CO₂ comprises at least 50% by weight of CO₂ in the liquid or supercritical state. According to another embodiment, the liquid formulation comprising the CO₂ comprises at least 60%, at least 70%, at least 80%, at least 85%, at least 90%, at least 95% or even at least 98% by weight of CO₂, with respect to the total weight of the liquid formulation comprising the CO₂. The generation of new molecules during the method according to the present invention is likely to modify the physico-chemical characteristics of the liquid formulation containing the CO₂. The latter can thus pass, at least partially, from the supercritical state to the liquid state, and vice versa. Preferentially, the CO₂ is in supercritical form.

According to one embodiment of the present invention, the liquid formulation comprising the CO₂ may further comprise a solvent in a minority amount with respect to the CO₂ in the liquid or supercritical state, i.e. less than 50% by weight with respect to the total weight of the liquid formulation comprising the CO₂. Such a solvent, when present, must necessarily be soluble with the supercritical CO₂. It can especially be water, an alcohol, especially methanol, isopropanol or ethanol, or a mixture thereof. For example, the liquid formulation comprising the CO₂ may comprise CO₂ in liquid or supercritical phase, water as solvent and methanol as co-solvent. According to one embodiment, the liquid formulation comprising the CO₂ comprises, or consists of, at least 50% by weight of CO₂ in the liquid or supercritical phase, and less than 50% by weight of water and/or methanol.

According to one embodiment of the present invention, the liquid formulation comprising the CO₂ comprises less than 40% by weight of solvent(s) with respect to total weight of the liquid formulation comprising the CO₂, less than 30%, less than 20%, less than 15%, less than 10%, less than 8% or less than 5% by weight of solvent(s) with respect to the total weight of the liquid formulation comprising the CO₂. The solvent is then preferentially water, methanol or a mixture of the two.

At the cathode, the CO₂ is reduced to CO or potentially to other forms such as methanol. The electrochemical reduction of CO₂ leads to CO, which can then be a potential source of energy, whether in simple molecules like methanol or methane, or in more complex molecules such as glucose or hydrocarbons.

At the cathode, in the presence of water, the reduction of H⁺ protons generates dihydrogen H₂ in a secondary reaction. The mixture of CO and H₂ (and residual CO₂) forms a gas mixture referred to as syngas, which is very useful for the synthesis of key chemical intermediates such as methanol and hydrocarbons. The Fischer-Tropsch method makes it possible to convert the syngas into various hydrocarbons and fuel oil. Today, methanol is synthesized almost exclusively from syngas using a method similar to the Fischer-Tropsch method, according to the following equation: CO+2H₂→CH₃OH.

According to one embodiment, the reduction method is carried out in the absence of an electrolyte, or in the presence of an amount of less than 5% by weight with respect to the total weight of the liquid (or liquid formulation) comprising the carbon dioxide, for example in the presence of an amount of less than 4%, in the presence of an amount of less than 3%, in the presence of an amount of less than 2%, in the presence of an amount of less than 1%, or even in the presence of an amount of less than 0.5%. According to another embodiment, the reduction method is carried out in the absence of catalyst or in the presence of an amount of catalyst of less than 10% by weight, with respect to total weight of the liquid (or liquid formulation) comprising the carbon dioxide, for example in the presence of an amount of less than 5%, in the presence of an amount of less than 4%, in the presence of an amount of less than 3%, in the presence of an amount of less than 2%, in the presence of an amount of less than 1%, or even in the presence of an amount of less than 0.5%. Thus, the method of the present invention is preferably electrolyte-free (or contains minimal amounts thereof) and/or catalyst-free (or contains minimal amounts thereof), which considerably reduces the cost of the method of the present invention. Indeed, it has been discovered that the short distance between the electrodes does away with the need for electrolyte and/or catalyst.

The method of the present invention can especially be carried out according to the following embodiments:
said reduction method is carried out in the presence of a proton donor compound, such as water or demineralized water, preferentially present at the anode;
said reduction method is carried out in the presence of water, preferentially present at the anode;
said reduction method is carried out in the presence of water, preferentially present at the anode or only present at the anode;
said reduction method is carried out under milli-fluidic or micro-fluidic conditions;
said method does not comprise the use of an electrolysis membrane;
said method comprises the use of an electrolysis membrane, such as an ion exchange membrane, preferentially a proton exchange membrane;
the carbon dioxide (i.e. the carbon dioxide entering the method) in the liquid or supercritical state comprises a solvent in a minority amount with respect to the CO₂ in the liquid or supercritical state, for example water or an alcohol, especially less than 30% by weight of water with respect to the total weight of the liquid entering the method, preferentially in an amount of less than or equal to 15% water, in an amount of less than or equal to 10% water by weight with respect to the total weight of the liquid entering the method, in particular in the presence of an ion exchange membrane;
water is only present at the anode (in this case, the electrodes are separated by a membrane);
said method comprises the following steps:
   a. a step of pressurizing CO₂ in the presence of water,
   b. applying a voltage comprised between 0.1 volts and 200 volts, preferentially between 1 and 10 volts, between said at least two electrodes,
   c. optionally checking the progress of the reaction, and
   d. recovering products resulting from the reduction reaction;
the reduction reaction can be carried out until obtaining carbon monoxide (CO);
the reduction reaction is carried out until obtaining at least one hydrocarbon product, such as a carboxylic acid, aldehyde, ketone, alcohol, alkane and/or alkene; and/or
the reduction reaction is carried out until obtaining at least one reduced product, such as carbon monoxide (CO) and/or a hydrocarbon product, such as a carboxylic acid, an aldehyde, a ketone, an alcohol, an alkane, an alkene and/or a mixture of these compounds.

The method of the invention can be carried out with or without an ion exchange membrane (such as a proton exchange membrane).

In one embodiment, the subject matter of the present invention relates to a method for the electrochemical reduction of CO₂ in the liquid or supercritical state with an ion exchange membrane (such as a proton exchange membrane) and comprising at least two electrodes separated from each other by a distance of less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 150 μm, such as less than or equal to 100 μm.

Advantageously, the subject matter of the present invention relates to a method for the electrochemical reduction of CO₂ in the liquid or supercritical state with an ion exchange membrane (such as a proton exchange membrane) and comprising at least two electrodes separated from each other by a distance of less than or equal to 600 μm, less than or equal to 500 μm, less than or equal to 400 μm, less than or equal to 300 μm, less than or equal to 200 μm, preferentially comprised between 200 and 400 μm.

In one embodiment, the subject matter of the present invention relates to a method for the electrochemical reduction of CO₂ in the liquid or supercritical state without an ion exchange membrane (such as a proton exchange membrane) comprising at least two electrodes separated from each other by a distance of less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 100 μm, such as less than or equal to 50 or 40 μm.

Advantageously, the subject matter of the present invention relates to a method for the electrochemical reduction of CO₂ in the liquid or supercritical state without an ion exchange membrane (such as a proton exchange membrane) comprising at least two electrodes separated from each other by a distance of less than or equal to 800 μm, less than or equal to 700 μm, less than or equal to 600 μm, less than or equal to 500 μm, less than or equal to 400 μm, such as comprised between 500 and 600 μm.

When using a reactor with a proton exchange membrane (PEM), the membrane can be separated from the electrodes by meshes or fabrics.

Preferably, a metal mesh—for example made of 316 stainless steel—separates the anode (the electrode connected to the positive pole of the generator) and the PEM membrane. This mesh allows the conducting of current and the passage of proton-donor fluids. A chemically neutral polymer mesh (of a polymer such as polypropylene) separates the cathode (electrode connected to the negative pole of the generator) and the PEM membrane. This mesh is an electrical insulator, but allows the passage of fluids: the carbon dioxide in liquid and/or supercritical phase.

The choice of a membrane applicable to the method according to the present invention can be made according to several criteria.

For example, the membrane is preferentially designed for water electrolysis; indeed, the same reaction of oxidation of water H₂O into O₂ and H⁺ can take place at the anode (water is then chosen as the proton donor). The membrane is therefore preferentially proton-conducting (H⁺) and/or advantageously impermeable to gases (e.g. it prevents the mixing of gases).

The membrane is preferentially reinforced. The reinforcement is advantageous for withstanding pressure variations and differences (in particular when there are variations or differences between the compartments on either side of the membrane).

The thickness of the membrane is adjusted based on the desired properties. Thus, the membrane is preferentially thicker if it is sought to avoid fluid contamination (the increase of the thickness limits the passage of fluids). Alternatively, the membrane is preferentially thinner if it is sought to increase the reduction performance (the lowering of the thickness facilitates the passage of protons).

Advantageously, the ion exchange membranes can be fluorinated, such as PTFE (polytetrafluoroethylene) or PFSA (perfluorosulfonic acid).

Advantageously, the ion exchange membranes can comprise a reinforcement in order, for example, to better withstand pressure variations (e.g. up to 15 bar). The ion exchange membranes can thus comprise a PEEK (PolyEther Ether Ketone) reinforcement.

Thus, a so-called Nafion™ membrane, in particular Nafion™ 115, (of the sulfonated tetrafluoroethylene type), which can be used, for example, to separate the anode and cathode compartments of fuel cells with a proton exchange membrane or in the context of water electrolyzers, may be entirely suitable for the method according to the present invention. The Nafion™ 115 cation exchange membrane has a thickness of 127 μm (5 mil).

Fumasep-type membranes, such as Fumasep F-1075-PK with a thickness of 75 μm, or Fumasep F-10120-PK with a thickness of 120 μm, can also be used. On the other hand, there has been a marked improvement in the stability of these membranes in recent years, which makes them increasingly suitable for a wide range of applications (in particular according to the present invention).

However, a PEEK reinforcement can advantageously be chosen for fluorinated membranes, such as PTFE or PFSA, in particular for the Fumasep cation exchange membranes (Fumasep F-1075-PK or Fumasep F-10120-PK).

According to one embodiment of the present invention, at least one of the two electrodes is selected from an electrode comprising copper, gold, silver, zinc, palladium, chromium, aluminum, carbon (such as graphite, graphene or carbon fibers), gallium, lead, mercury, graphite, indium, tin, cadmium, thallium, nickel, iron, platinum, titanium and alloys thereof, such as steel or stainless steel, and such as brass (copper-zinc alloy). For example, at least one of the two electrodes is selected from an electrode made of copper, gold, silver, zinc, palladium, chromium, aluminum, carbon (such as graphite, graphene or carbon fibers), gallium, lead, mercury, graphite, indium, tin, cadmium, thallium, nickel, iron, platinum, titanium and alloys thereof, lead, mercury, graphite, indium, tin, cadmium, thallium, nickel, iron, platinum, titanium and alloys thereof, such as steel or stainless steel, and such as brass (copper-zinc alloy). Preferably, at least one electrode is selected from an electrode comprising copper, platinum, chromium, carbon (such as graphite), zinc, iron, or an alloy thereof (such as steel or stainless steel). For example, at least one of the two electrodes is a copper electrode, for example a copper anode or cathode. According to another example, at least one of the two electrodes is an iron electrode, for example an iron anode or cathode. In yet another example, at least one of the two electrodes is a zinc electrode, for example a zinc anode or cathode. In yet another example, at least one of the two electrodes is a carbon electrode, for example a carbon anode or cathode.

According to one preferred embodiment of the present invention, at least one of the two electrodes is a steel electrode, for example an anode or a cathode made of stainless steel, such as 316 stainless steel.

Preferably, the electrodes used in the method according to the present invention comprise at least 50% by weight of a metal (such as copper) or of a metal alloy having a degree of oxidation 0 (i.e. in purely metallic form), preferentially at least 60% by weight, at least 70% by weight, at least 80% by weight, at least 85% by weight, at least 90% by weight, at least 95% by weight or even at least 98% by weight of a metal (such as copper) or of a metal alloy having a degree of oxidation 0, with respect to the total weight of the electrode. More preferably, the electrodes used in the method according to the present invention comprise at least 99% by weight of a metal (such as copper) or of a metal alloy having a degree of oxidation 0.

According to one preferred embodiment of the present invention, at least one electrode is selected from an electrode comprising copper and/or iron or an alloy thereof.

According to another preferred embodiment of the present invention, one of the at least two electrodes comprises iron or an alloy thereof and another of the at least two electrodes comprises copper.

According to certain particular embodiments which may be combined with each other:
the cathode comprises copper and the anode comprises platinum or graphite;
the cathode comprises copper and the anode comprises platinum or is made of platinum;
the cathode comprises copper and the anode comprises graphite or is made of graphite;
the cathode comprises copper and the anode comprises iron;
the cathode comprises copper and the anode comprises steel, stainless steel such as 316 stainless steel;
the cathode is made of copper and the anode is made of iron; or
the cathode is made of copper and the anode is made of steel, such as 316 stainless steel.

In one particular embodiment, the reaction for electrochemical reduction of CO₂ in the liquid or supercritical state of the method according to the present invention is carried out at a pressure greater than or equal to 60 bar, greater than or equal to 70 bar, greater than or equal to 75 bar, greater than or equal to 80 bar, greater than or equal to 85 bar, greater than or equal to 90 bar, greater than or equal to 95 bar, greater than or equal to 100 bar, greater than or equal to 105 bar, greater than or equal to 110 bar, greater than or equal to 115 bar, greater than or equal to 120 bar, greater than or equal to 125 bar, greater than or equal to 130 bar, greater than or equal to 135 bar, greater than or equal to 140 bar, greater than or equal to 145 bar, greater than or equal to 150 bar, greater than or equal to 155 bar, greater than or equal to 160 bar or even greater than or equal to 165 bar.

In another particular embodiment, the reaction for electrochemical reduction of CO₂ in the liquid or supercritical state of the method according to the present invention is carried out at a temperature greater than or equal to 0° C., greater than or equal to 5° C., greater than or equal to 10° C., greater than or equal to 15° C., greater than or equal to 20° C., greater than or equal to 25° C., greater than or equal to 30° C., greater than or equal to 35° C., greater than or equal to 40° C., greater than or equal to 45° C., greater than or equal to 50° C., or even greater than or equal to 60° C.

Preferably, the reaction for electrochemical reduction of CO₂ in the liquid or supercritical state of the method according to the present invention is carried out at a temperature comprised between 30° C. and 60° C., preferentially comprised between 35° C. and 55° C., more preferentially comprised between 40° C. and 50° C., such as 44° C. plus or minus 3° C.

Preferably, the reaction for electrochemical reduction of CO₂ in the liquid or supercritical state of the method according to the present invention is carried out at a temperature greater than or equal to 0° C. and at a pressure greater than or equal to 50 bar.

More preferably, the reaction for electrochemical reduction of CO₂ in the liquid or supercritical state of the method according to the present invention is carried out at a temperature greater than or equal to 20° C., or even greater than or equal to 30° C., and at a pressure greater than or equal to 70 bar, or even greater than or equal to 80 bar.

Even more preferably, the reaction for electrochemical reduction of CO₂ in the liquid or supercritical state of the method according to the present invention is carried out at a temperature greater than or equal to 35° C., or even greater than or equal to 40° C., and at a pressure greater than or equal to 90 bar, or even greater than or equal to 100 bar.

In one embodiment, the reaction for electrochemical reduction of CO₂ in the liquid or supercritical state of the method according to the present invention is carried out at a pH greater than or equal to 2, greater than or equal to 4, greater than or equal to 5, greater than or equal to 6, greater than or equal to 7, greater than or equal to 8, greater than or equal to 9, greater than or equal to 10, greater than or equal to 11 or even greater than or equal to 12.

In one particular embodiment, the reaction for electrochemical reduction of CO₂ is carried out under milli-fluidic or micro-fluidic conditions, involving a small distance between the electrodes, and optionally a continuous fluid flow within the device. The conditions under which the reduction method is carried out can thus be arranged in such a way that a carbon dioxide film with a thickness of less than or equal to 5 mm, less than or equal to 4 mm, less than or equal to 3 mm, less than or equal to 2 mm, less than or equal to 1 mm, preferentially less than or equal to 500 microns (μm), less than or equal to 400 microns, less than or equal to 300 microns, less than or equal to 200 microns, less than or equal to 150 microns, or even less than or equal to 100 microns, is generated.

In one particular embodiment, the reaction for electrochemical reduction of carbon dioxide in the liquid or supercritical state of the method according to the present invention is carried out in the context of an industrial production, i.e. with amounts of product(s) obtained greater than or equal to 10 kg, greater than or equal to 25 kg, greater than or equal to 50 kg, greater than or equal to 100 kg, greater than or equal to 500 kg, greater than or equal to 1,000 kg, greater than or equal to 5,000 kg, or even greater than or equal to 10,000 kg, per day.

In one particular embodiment, the method for the electrochemical reduction of CO₂ in the liquid or supercritical state of the method according to the present invention is carried out in the presence of water, in particular in the absence of an ion exchange membrane. The absence of membrane combined with the absence of electrolyte makes the method according to the present invention particularly attractive in terms of industrialization. Furthermore, in this configuration, it is advantageous to use a continuous (flow) reactor, such as a planar or tubular reactor. In fact, this type of reactor allows easy and completely safe industrialization, by avoiding generating uncontrolled pressure surges.

In the case that a membrane is present, the presence of water on the anode side in particular allows the reduction of the CO on the cathode side. The anodic solution may comprise an amount of water or a proton-donor equivalent greater than or equal to 10% by weight, greater than or equal to 25% by weight, greater than or equal to 50% by weight, greater than or equal to 75% by weight, greater than or equal to 80% by weight, greater than or equal to 90% by weight or even greater than or equal to 95% by weight of water, with respect to the total weight of the anodic solution.

In the absence of membrane, the presence of water or a proton-donor equivalent in the carbon dioxide in the liquid or supercritical state may be less than or equal to 20% by weight, less than or equal to 15% by weight, less than or equal to 10% by weight or even less than or equal to 5% by weight of water, with respect to the total weight of the solution.

In one embodiment of the present invention, the water used, or a proton-donor equivalent, has a conductivity (20° C.) of less than or equal to 40 μS/cm, less than or equal to 30 μS/cm, less than or equal to 20 μS/cm, less than or equal to 10 μS/cm, less than or equal to 9 μS/cm, less than or equal to 8 μS/cm, less than or equal to 7 μS/cm, less than or equal to 6 μS/cm or even less than or equal to 5 μS/cm.

In the context of the present invention, two designs of reactor or electrochemical cell are possible: with an ion exchange membrane (in particular a proton exchange membrane) or without a membrane. When the reactor comprises an ion exchange membrane (in particular a proton exchange membrane), the simplest embodiment consists in using a pump to circulate a reducing liquid (in particular water) in the anode compartment (oxidation site) and CO₂ in the supercritical phase in the cathode compartment; in this case, the CO₂ in the supercritical phase is electrically conductive without adding electrolytes. When the reactor does not comprise an ion exchange membrane, according to one embodiment, an amount of water of less than 5% by weight can be, for example, mixed with the CO₂ in the supercritical phase.

Additionally, depending on the voltage applied, it is possible to orient the chemical reactions by selecting threshold effects below which some reactions would be minimized with respect to other reactions, for example.

The method for the electrochemical reduction of CO₂ in the liquid or supercritical state according to the present invention can comprise a voltage comprised between 0.1 volts and 200 volts, between 1 and 50 volts, between 2 and 25 volts, between 3 and 15 volts, between 4 and 10 volts, between 5 and 9 volts, or even between 6 and 8 volts, such as 7 volts plus or minus 0.5 volts between the electrodes.

In one particular embodiment, the method for the electrochemical reduction of CO₂ in the liquid or supercritical state according to the present invention may comprise a voltage of less than or equal to 40 volts, less than or equal to 36 volts, less than or equal to 24 volts, less than or equal to 12 volts, less than or equal to 9 volts or even less than or equal to 5 volts.

In one particular embodiment, it may be advantageous to adjust the voltage according to the distance between the electrodes and according to the constituents to be electrolyzed (water, for example, requires a minimum voltage of 1.23 volts).

For example, for a voltage of less than or equal to 100 volts, the distance between the at least two electrodes may be less than or equal to 7 mm, such as less than or equal to 1 mm, less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 150 μm, less than or equal to 100 μm, such as less than or equal to 50 or 40 μm.

For example, for a voltage of less than or equal to 40 volts, the distance between the at least two electrodes may be less than or equal to 5 millimeters, such as less than or equal to 1 mm, less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 150 μm, less than or equal to 100 μm, such as less than or equal to 50 or 40 μm.

For example, for a voltage of less than or equal to 24 volts, the distance between the at least two electrodes may be less than or equal to 2 mm, such as less than or equal to 1 mm, less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 150 μm, less than or equal to 100 μm, such as less than or equal to 50 or 40 μm.

For example, for a voltage comprised between 5 volts and 9 volts, the distance between the at least two electrodes may be less than or equal to 1 mm, such as less than or equal to 900 μm, less than or equal to 800 μm, less than or equal to 700 μm, less than or equal to 600 μm, less than or equal to 500 μm, less than or equal to 400 μm, less than or equal to 300 μm, less than or equal to 200 μm, less than or equal to 150 μm, less than or equal to 100 μm, such as less than or equal to 50 or 40 μm.

The method for the electrochemical reduction of carbon dioxide in the liquid or supercritical state according to the present invention may comprise a current comprised between 0.1 mA·cm⁻² and 1 A·cm⁻², between 1 mA·cm⁻² and 500 mA·cm⁻², between 5 mA·cm⁻² and 250 mA·cm⁻², between 10 mA·cm⁻² and 100 mA·cm⁻², between 25 mA·cm⁻² and 85 mA·cm⁻², between 30 mA·cm⁻² and 70 mA·cm⁻², between 40 mA·cm⁻² and 60 mA·cm⁻² or even between 45 mA·cm⁻² and 50 mA·cm⁻², such as 50 plus or minus 2 mA·cm⁻² between the electrodes.

In the various embodiments of the present invention described herein, the reduction reaction can further be controlled easily based on the reaction time.

“Reaction time” is understood in the context of the present invention to mean the residence time in the reactor, between the energized electrodes. Synonyms for “reaction time” might be “residence time” or “contact time”. These expressions are therefore interchangeable.

Any type of inspection test/analysis that makes it possible to assess the progress of the reaction and thus identify the species produced can thus be applied.

Thus, the reduction reaction can be carried out until obtaining CO and/or a hydrocarbon product, such as a carboxylic acid, aldehyde, ketone, alcohol, alkane and/or alkene.

The reaction times can, for example, vary between 0.1 minutes and one hour, preferentially between 0.2 minutes and 30 minutes, between 0.5 minutes and 10 minutes, between 1 minute and 5 minutes, between 2 minutes and 4 minutes, or even between 3 minutes and 4 minutes.

Preferentially, the reaction times can vary between 0.12 minutes and 9 minutes, between 0.14 minutes and 8 minutes, between 0.16 minutes and 7 minutes, between 0.18 minutes and 6 minutes, or even between 0.2 minutes and 4 minutes.

In one particular embodiment:
the voltage is comprised between 5 volts and 9 volts;
the intensity is comprised between 30 mA·cm⁻² and 70 mA·cm⁻²;
the distance between the electrodes is comprised between 200 and 400 μm in the configuration with membrane or comprised between 500 and 600 μm in the configuration without membrane;
the pressure is comprised between 75 and 125 bar;
the reactor temperature is comprised between 40° C. and 60° C.;
preferentially, the carbon dioxide is in supercritical form (i.e. 31.06° C. and 73.83 bar);
the water used has a conductivity of less than or equal to 10 μS/cm;
the average residence time of the CO₂ in the reactor is between 0.2 and 4 minutes; and/or
the mixture of water/scCO₂ (liquid equivalent; “scCO₂” for supercritical carbon dioxide), is comprised between (0.20 and 0.50) of water and (0.50 and 0.80) of scCO₂, respectively, in the case where the membrane is absent.

Any technique for recovering products resulting from the reduction reaction is applicable. In particular, any industrial technique for recovering products resulting from the reduction reaction is particularly preferred. For example, the products may be compressed, expanded, heated or cooled, depending on the boiling temperatures and pressures of the desired compounds. Alternatively, or in combination, liquid and gas chromatographic purification techniques can be used.

A second subject of the present invention also relates to a reactor, preferentially an industrial reactor, for carrying out the method for the electrochemical reduction of carbon dioxide according to the present invention, as described hereinbefore. Such a reactor comprises at least two electrodes separated from each other by a distance of less than or equal to 7 mm.

The reactor according to the present invention is capable of withstanding pressures greater than 73.9 bar (72.9 atm) and wherein the fluids flow in layers of micrometric or millimetric thickness.

The reactor, as well as the entering fluids, are preferentially maintained at a temperature greater than 32° C. to enable the CO₂ to be and remain in the supercritical state.

Additionally, such a reactor may especially have the following optional features:
said reactor operates in continuous (flow), preferentially in tubular and/or planar form; and/or
said reactor is suitable for withstanding pressures greater than or equal to 74 bar, for example 80 bar, 90 bar or 100 bar.

The continuous flow of the fluids (water, CO₂, etc.) is a characteristic of a so-called “continuous” method. The flow rate of the CO₂, or of the water/CO₂ mixture in the case of the reactor without membrane, determines the residence time for the CO₂ in the reactor, i.e. the contact time of the CO₂ with the electrode, i.e. the duration of the reaction.

However, it is also possible to apply, in the context of the present invention, a discontinuous or sequential method wherein the fluids are immobilized in the reaction chamber for a predefined contact time and then replaced sequentially. It is also possible to design continuous reactors that consist of several cylinders nested inside one another. According to this configuration, the notion of contact time is used (rather than the notion of fluid flow rate).

When the method is of the discontinuous type, the average contact time used in the reactor may be less than or equal to one hour, less than or equal to 30 minutes, less than or equal to 20 minutes, less than or equal to 15 minutes, less than or equal to 10 minutes, less than or equal to 9 minutes, less than or equal to 8 minutes, less than or equal to 7 minutes, less than or equal to 6 minutes, less than or equal to 5 minutes, less than or equal to 4 minutes, less than or equal to 3 minutes, less than or equal to 2 minutes, less than or equal to 1 minute.

The average contact time used in the reactor can thus be comprised between 0.1 minutes (min) and 1 hour (h), preferentially between 0.2 min and 30 min, between 0.5 min and 10 min, between 1 min and 5 min, between 2 min and 4 min, or even between 3 min and 4 min.

Preferably, the average contact time used in the reactor can be comprised between 10 seconds(s) and 5 min.

In a preferred embodiment, said reactor is suitable for withstanding high pressures.

More particularly, the reactor can consist of several hollow and/or grooved machined parts (e.g. four machined parts) of the same shape (e.g. polygonal such as a square) so that these parts can be “sandwiched” together. At two opposite ends of the sandwich, two metal parts (e.g. made of steel) of a thickness making it possible to withstand the pressures applied are clamped together by several bolts (such as eight stainless-steel bolts ensuring that high pressures are withstood). At the middle of the sandwich, two plastic parts house the electrodes and the flow of fluids: water and carbon dioxide in the supercritical state. The electrodes can, for example, be circular. Gaskets ensure the tightness of the system.

A means of electrical contact, such as a wire, is inserted into the reactor in order to make it possible to supply electrical power to the electrodes.

Advantageously, a reactor comprising a tubular system can easily be subjected to high pressures.

“Tubular system” or “tubular device” (equivalent expressions) is understood to mean a system (or device) comprising at least one hollow body through which a fluid can pass.

Electrodes and optionally at least one ion exchange membrane (in particular a cation exchange membrane) can be inserted into such a tubular system. It is thus possible, for example, to design continuous reactors consisting of at least one cylinder nested inside another, each cylinder making it possible to perform a specific function (membrane, electrode, shell to isolate the system from the outside environment).

There are several advantages to such a system: for example, as the pressure is distributed over a large tube surface, the risk of explosion (i.e. a violent and dangerous deflagration) is limited. Another advantage of such a system is the possibility of a long tube length, allowing a high fluid flow rate to be applied while exposing the fluid to a desired reaction time (proportional to the length of the tube).

One subject matter of the present invention also relates, generically, to a reactor, preferentially an industrial reactor, for carrying out a method for the electrochemical reduction of CO₂ in the critical or supercritical state, such a reactor comprising at least two electrodes separated from each other by a distance of less than or equal to 7 mm, for example of less than or equal to 5 mm, less than or equal to 3 mm, less than or equal to 1 mm.

A third subject of the present invention relates to a method for synthesizing methanol and/or at least one hydrocarbon from CO and H₂, the CO being obtained according to the method for the electrochemical reduction of CO₂ of the present invention. The method for synthesizing at least one hydrocarbon may especially consist in carrying out a Fischer-Tropsch reaction.

A fourth subject of the present invention also relates to an industrial reactor complement, or a second reactor positioned in series with the first, for carrying out the method for the electrochemical reduction of CO₂ according to the present invention for carrying out the method for synthesizing methanol and/or at least one hydrocarbon from CO obtained according to the method for the electrochemical reduction of carbon dioxide described herein. The method for synthesizing at least one hydrocarbon may especially consist in carrying out a Fischer-Tropsch reaction.

A fifth subject of the present invention relates to a reaction device comprising at least one reactor for the electrochemical reduction of carbon dioxide in the liquid or supercritical state, such a reactor comprising at least two electrodes separated from each other by a distance of less than or equal to 7 mm, for example of less than or equal to 5 mm, less than or equal to 3 mm, less than or equal to 1 mm; for example, the reaction device according to the invention comprises one or more reactor(s) arranged in series and/or in parallel. The reaction device may, additionally, comprise an industrial reactor complement.

The method of the present invention, via the reaction device of the present invention, is scaled up to industrial scale by multiplying the initial reactor—in other words, by multiplying the initial installation. This scaling up to industrial scale offers many advantages, especially in terms of cost and technical difficulties, compared with a method that does not belong to the milli-fluidic or micro-fluidic context. Indeed, in this case, the method is scaled up to industrial scale by enlarging the installations, which calls for extensive process engineering studies.

A sixth subject of the present invention relates to the use of a reaction device comprising at least one reactor comprising at least two electrodes separated from each other by a distance of less than or equal to 7 mm, for example of less than or equal to 5 mm, less than or equal to 3 mm, less than or equal to 1 mm, for example comprising one or more reactor(s) arranged in series, for the electrochemical reduction of carbon dioxide in the liquid or supercritical state.

1. A method for the electrochemical reduction of carbon dioxide (CO₂) in the liquid or supercritical state, comprising at least two electrodes separated from each other by a distance of less than or equal to 7 millimeters.

2. The method according to claim 1, wherein the CO₂ in the liquid or supercritical state is in the form of a liquid formulation comprising at least 50% by weight of CO₂ in the liquid or supercritical state, with respect to the total weight of the liquid formulation.

3. The method according to claim 2, wherein the liquid formulation comprises no electrolyte or comprises an amount of electrolyte of less than 5% by weight, with respect to total weight of the liquid formulation.

4. The method according to claim 1, wherein at least one electrode comprises copper, gold, silver, zinc, palladium, chromium, aluminum, carbon (such as graphite), gallium, lead, mercury, graphite, indium, tin, cadmium, thallium, nickel, iron, platinum, titanium, steel, stainless steel and/or brass.

5. The method according to claim 1, characterized in that the reduction method is carried out in the presence of water, preferentially present at the anode or present only at the anode.

6. The method according to claim 1, characterized in that said method does not comprise the use of an electrolysis membrane.

7. The method according to claim 1, characterized in that said method comprises the use of an electrolysis membrane, such as an ion exchange membrane, preferentially a proton exchange membrane.

8. The method according to claim 1, characterized in that said method comprises the following steps:
a. a step of pressurizing CO₂ in the presence of water,
b. applying a voltage comprised between 0.1 volts and 200 volts, preferentially between 1 and 10 volts, between said at least two electrodes,
c. optionally checking the progress of the reaction, and
d. recovering products resulting from the reduction reaction.

9. The method according to claim 1, characterized in that the reduction reaction is carried out until obtaining at least one reduced product such as carbon monoxide (CO) and/or a hydrocarbon product, such as a carboxylic acid, an aldehyde, a ketone, an alcohol, an alkane and/or an alkene.

10. A method for synthesizing at least one hydrocarbon, consisting in carrying out a Fischer-Tropsch reaction from carbon monoxide (CO) and hydrogen (H₂), the CO being obtained according to the method of claim 1.

11-13. (Canceled)

14. A reaction device comprising at least one reactor for the electrochemical reduction of CO₂ in the liquid or supercritical state, said reactor comprising at least two electrodes separated from each other by a distance of less than or equal to 7 millimeters.

15. The device according to claim 14, comprising at least two reactors arranged in series.

16. The device according to claim 14, wherein the at least one reactor is of the continuous (flow) type.

17-18. (canceled)
